(19) United States
(12) Patent Application Publication
(10) Pub. No.: US 2026/0345210 A1
(43) Pub. Date: Nov. 5, 2026

(54) SENSORIZED INTERNAL FIXATOR AND RELATED MONITORING APPARATUS FOR EPIPHYSIODESIS INTERVENTION

(71) Applicant: Orthofix S.R.L., Bussolengo (VR) (IT)

(72) Inventors: Mario Donnici, Bussolengo (VR) (IT); Andrea Ottoboni, Giacciano con Baruchella (RO) (IT); Marco Modena, Verona (IT); Daniele Venturini, Povegliano (IT)

(21) Appl. No.: 19/488,274

(22) PCT Filed: Jul. 25, 2024
(86) PCT No.: PCT/EP2024/071163

§ 371 Date: Nov. 26, 2025

(30) Foreign Application Priority Data
Jul. 26, 2023 (IT) 102023000015768

(51) Int. Cl. A61B 17/80; A61B 5/00; A61B 17/00; A61B 17/86

(52) U.S. Cl. CPC A61B 17/8085; A61B 5/4851; A61B 17/8625; A61B 2017/00221; A61B 2017/00955

(57) ABSTRACT

An internal fixator device for epiphysiodesis intervention, comprising: a bone plate (20) comprising a pair of holding elements (21) with a respective through-hole (23) and a structural central portion (22); a pair of fixing screws (30), each comprising a respective stem (31) for fixing to a bone portion and a respective head (32) held in the through-hole (23). The central portion (22) is configured for a bending deformation of the bone plate (20) in the epiphysiodesis intervention. The heads (32) of the fixing screws (30) remain rotationally constrained to the respective holding elements (21), and the stems (31) of the fixing screws (30) are configured to spread apart defining an angle. The internal fixator device (10; 10′) further comprises at least one sensor (40; 40′) applied to the central portion (22) and configured to measure the bending deformation. The sensor (40; 40′) is further configured to wirelessly transmit data representing the bending deformation to calculate the angle defined by the stems (31) of the fixing screws (30).

### FIELD OF APPLICATION

The present invention relates to an internal fixator device to be used as correction means particularly for pediatric patients for epiphysiodesis intervention. The present invention also relates to a monitoring apparatus for epiphysiodesis intervention.

The invention finds useful application in the pediatric orthopedics field for epiphysiodesis interventions to the ends of deformed long bones of pediatric patients.

### PRIOR ART

Bone deformities are a problem especially encountered in the pediatric orthopedic practice. Pathological deformities, particularly of the lower limbs, may cause pain related to the biomechanical alteration of gait and joint instability in children and young people, and these effects are further exacerbated during the periods of growth.

In pediatric subjects who have physis (i.e. growth plates) that are not yet fused and therefore bones still growing, it is possible to avoid significant surgical interventions (such as osteotomy and internal/external fixation) by exploiting the modulation of growth by means of techniques capable of correcting the deformity over time through gradual bone remodeling. This approach, called epiphysiodesis or hemi-epiphysiodesis, may be achieved by using transphyseal screws, staples or bone plates on one side of the physis.

Among the techniques used for epiphysiodesis, blocking of the growth cartilage through the use of a bone plate is very common. Such a bone plate is applied to the bone by means of fixing screws, which constrain it to the epiphysis and to the metaphysis, respectively, at the convex area of the bone deformation. Both metaphyseal and epiphyseal portions of the bone plate have at least one through-hole adapted to house one of the above fixing screws.

When the fixing screws of the bone plate are positioned above and below the growth cartilage, they must adapt to the curvature resulting from the constraint of the bone plate and to the variations that occur during growth.

Particular bone plates are therefore known, which are constrained to the bone by means of fixing screws which are likely to spread apart, in order to avoid excessive compression of the growth cartilage and to delicately guide the natural growth process of the physis and bones.

A solution described by document U.S. Pat. No. 8,029,507 B2 relates to a plate fixator device for the angular correction of bone deformations, which comprises a pivoting hinge arranged transversally in the plate, to allow a relative rotation of the ends of the plate under the effect of the load of the fixing screws, so as to accommodate the curvature variations that occur during growth.

However, the solution known from document U.S. Pat. No. 8,029,507 B2 has large dimensions due to the presence of the hinge, which can reduce the tolerability of the bone plate especially in regions poorly covered by soft tissues. Moreover, the solution known from document U.S. Pat. No. 8,029,507 B2 does not allow having an adequate centering reference on the growth cartilage, for the use of a Kirschner wire for temporary fixation of the bone plate, according to known surgical techniques.

A further solution described in document U.S. Pat. No. 2004/0111089 A1 relates to a plate fixator device for controlling the bone development, in particular for correcting bones deformities; this device comprises two fixing screws which are inserted in proper holes obtained in the bone plate.

In an example described in U.S. Pat. No. 2004/0111089 A1, the head of each fixing screw has a spherical profile to suitably rotate in an elongated seat obtained in the plate, and thus to allow the fixing screws to spread apart. To allow a rotation of the stem of the screws, a spherical profile of the screw head is required, which also contains the engagement through which to operate the screw during tightening; therefore, in this known example, the screw head and in general the bone plate have large height dimensions, which can reduce the tolerability of the bone plate, especially in regions poorly covered by soft tissues.

In further examples described in US 2004/0111089 A1, it is provided to make a central connection between the holes of the bone plate flexible, so that the fixing screws are able to move following the movement of the bone sections. Examples of flexible connections known from U.S. Pat. No. 2004/0111089 A1 provide for: a midsection of the plate made of flexible material, a plate made of fabric with rigid material surrounding the openings, or the use of a flexible band to surround the fixing screws instead of a plate, possibly including a crimp in the center.

Still a solution of bone plate for epiphysiodesis is described in document WO 2018/134319 A2, which relates to an internal fixing device comprising a pair of holding elements, each respectively comprising a through-hole to receive a respective fixing screw to a bone; the internal fixator device further comprises a central portion which structurally connects and constrains the holding elements to each other; the central portion is flexible so as to allow the bone plate to bend; each through-hole comprises a threaded surface adapted to couple with a corresponding surface of the respective fixing screw; the holding elements and the central portion are provided as a composite structure. Although the implants known in the art partially improve the tolerability of the bone plate for the patient, allowing the fixing screws to accommodate the variations that occur during growth, nevertheless they have some limitations.

In particular, the spreading apart of the fixing screws during the evolution of the epiphysiodesis or semi-epiphysiodesis intervention must be periodically monitored, to evaluate the clinical picture and the effectiveness of the bone remodeling intervention.

To evaluate the correction progress of the deformity provided by the bone plate, it is therefore necessary to subject the patient, of pediatric to juvenile age, to radiographic checks which however involve additional risks.

### Objects of the Invention

An object of the present invention is to solve problems of the prior art.

A particular object of the present invention is to allow tracing the configuration of the fixing screws of a bone plate for epiphysiodesis.

A further particular object of the present invention is to provide the surgeon with information on the configuration of the fixing screws and the bone plate.

A further particular object of the present invention is to integrate a solution into a more effective clinical context.

A further particular object of the present invention is to devise a bone plate implant that can be fixed to the bone in an optimal manner.

A further particular object of the present invention is to devise a bone plate implant which is better tolerable for the patient, even when applied in anatomical regions with poor soft tissue coverage.

A further particular object of the present invention is to devise a bone plate implant which is robust and suitable for epiphysiodesis applications.

A further particular object of the present invention is to devise a bone plate implant which does not interfere with the growth cartilage.

These and other objects are achieved by means of an internal fixator device and a monitoring apparatus for epiphysiodesis intervention according to the features of the enclosed claims, which are an integral part of the present description.

### SUMMARY OF THE INVENTION

A solution idea underlying the present invention is to provide an internal fixator device which comprises a bone plate and a pair of fixing screws.

The bone plate comprises a pair of holding elements and a central portion which structurally connects the pair of holding elements. Each of the holding elements comprises a respective through-hole.

Each of the fixing screws comprising a respective stem configured for fixing to a bone portion and a respective head configured to be held in the respective through-hole.

The central portion of the bone plate is configured for a bending deformation of the bone plate in the epiphysiodesis intervention.

The heads of the fixing screws remain rotationally constrained to the respective holding elements and the stems of the fixing screws are configured to spread apart, thus defining an angle during the bending deformation of the plate.

The internal fixator device further comprises at least one sensor, applied to the central portion of the plate. The at least one sensor is configured to measure the bending deformation of the plate. The at least one sensor is further configured to wirelessly transmit data representing the bending deformation, to calculate the angle defined by the stems.

A further solution idea underlying the present invention is to provide a monitoring apparatus for epiphysiodesis intervention. The monitoring apparatus comprises at least one internal fixator device according to the present invention. The monitoring apparatus comprises at least one reader configured to wirelessly receive data representing the bending deformation transmitted by at least one sensor. The monitoring apparatus comprises at least one processor configured to process the data representing the bending deformation to calculate the angle defined by the stems in a development of said epiphysiodesis intervention.

Preferably, the bone plate is made of a plastic material, in particular having an at least partially flexible central portion.

Advantageously, the present invention allows tracing the angular configuration of the fixing screws of the bone plate for epiphysiodesis, obtained in terms of deformation related to a bending of the plate itself. Advantageously, the present invention allows providing the surgeon, even from remote, with real-time information on the angular configuration of the fixing screws of the bone plate.

Advantageously, the present invention allows providing an information solution integrated within the bone plate, thus defining a more effective clinical context.

Advantageously, the present invention allows building an entire system or platform that can be used by surgeons expert in the treatment of bone deformities-especially in the young pediatric population-capable of actively providing information on the angular configuration of the fixing screws of the bone plate and/or on the deformation related to a bending of the plate itself, measured in a simpler way even in an outpatient or home context and in real time.

Advantageously, the present invention allows providing information on the configuration of the internal fixator device and evaluating corresponding changes in the bone deformity.

Advantageously, the present invention allows the surgeon to control the progress of the epiphysiodesis intervention more effectively.

Preferably, the central portion of the plate comprises a raised surface which defines a concavity with respect to bottom surfaces of the holding elements, facing towards the stems. Advantageously, the raised surface is thus adapted to house the at least one sensor. Advantageously, in this way the tolerability of the fixation device is improved by limiting pressure on the bone physis and avoiding lateral crushing of the growth cartilage.

Further features and advantages of the bone plate internal fixator device of the present invention will become clearer from the following description of exemplifying embodiments given by way of illustrative and non-limiting example.

### BRIEF DESCRIPTION OF THE DRAWINGS

The present description relates to the enclosed drawings, in which:

FIG. 1 illustrates a side view of a first embodiment of the internal fixator device for epiphysiodesis intervention.

FIG. 2 illustrates a perspective view of the internal fixator device of FIG. 1 in an undeformed condition.

FIG. 3 illustrates a perspective view of the internal fixator device of FIG. 2 in a flexurally deformed condition and with spread apart fixing screws.

FIG. 4 illustrates a bottom view of the internal fixator device of FIG. 1.

FIG. 5 illustrates a sectional view of the internal fixator device of FIG. 4.

FIG. 6 illustrates a perspective view of a second embodiment of the internal fixator device for epiphysiodesis intervention.

FIG. 7 exemplifies the operation of a monitoring apparatus for epiphysiodesis intervention according to the present invention.

In the different figures, similar elements will be indicated with similar reference numbers.

### DETAILED DESCRIPTION OF THE PREFERRED EMBODIMENTS

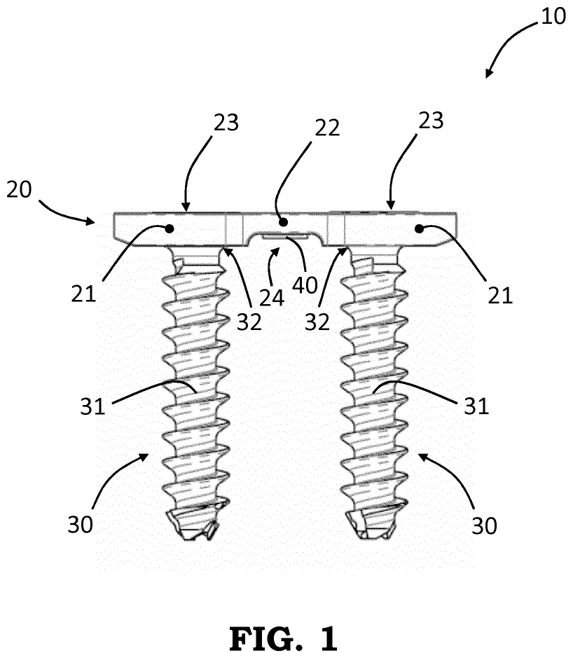

FIG. 1 illustrates a side view of a first embodiment of the internal fixator device 10.

The internal fixator device 10 is configured for an epiphysiodesis intervention, namely it is particularly adapted for use in orthopedics for the treatment of malformations of long bones, in particular for pediatric and/or adolescent patients, with application of the fixator device 10 across the physis of these bones.

The internal fixator device 10 comprises a bone plate 20, which comprises a pair of holding elements 21 and a central portion 22. The central portion 22 structurally connects the pair of holding elements 21. Each holding element 21 comprises a respective through-hole 23, which will be further described.

The holding elements 21 are preferably made of a rigid and biocompatible material and are substantially circular-shaped, of uniform thickness.

The internal fixator device 10 comprises a pair of fixing screws 30. Each fixing screw 30 comprising a respective stem 31 configured for fixing to a bone portion, in particular across a bone physis. Each fixing screw 30 further comprising a respective head 32 configured to be held in the respective through-hole 23.

As it will be further described, the central portion 22 is configured for a bending deformation of the bone plate 20, in the epiphysiodesis intervention.

Preferably, the bone plate 20 is made of a plastic material, preferably in a single piece. Preferably, the plastic material of the bone plate 20 comprises a thermoplastic organic polymer, in particular polyether ether ketone or PEEK. Indeed, in general, polyether ether ketone or PEEK has adequate resistance and biocompatibility features.

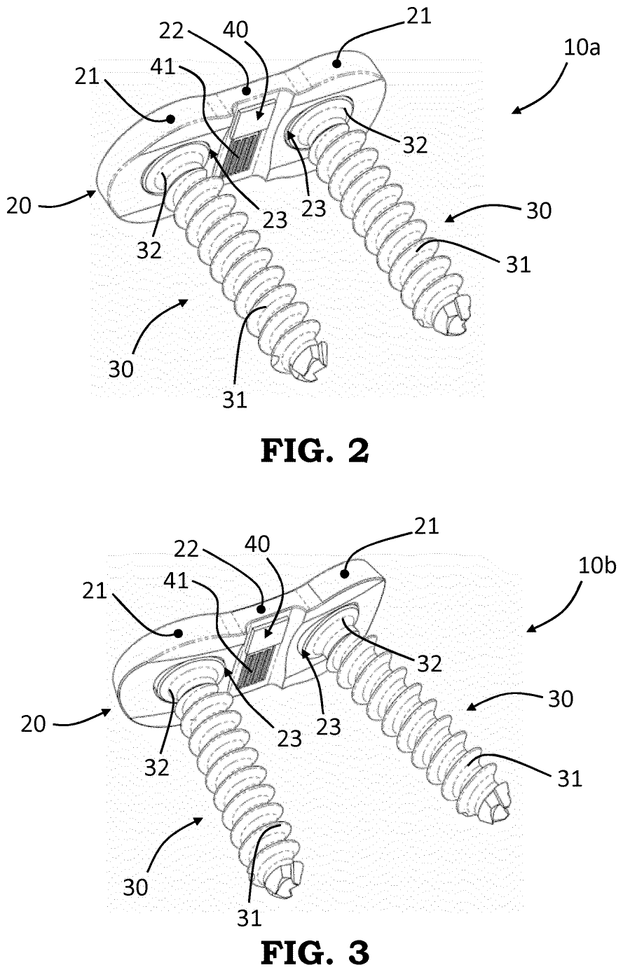

FIG. 2 illustrates a perspective view of the internal fixator device 10a corresponding to an undeformed condition of the internal fixator device 10, for instance corresponding to an initial implantation of the internal fixator device 10 for an epiphysiodesis intervention.

The internal fixator device 10a comprises the bone plate 20 with the pair of holding elements 21 and the central portion 22.

The internal fixator device 10a comprises the pair of fixing screws 30, each comprising the respective stem 31 and the respective head 32 held in the respective through-hole 23 of the plate 20.

Through the fixing screws 30, the holding elements 21 are thus adapted to be fixed to the epiphysis and metaphysis of a long bone of a pediatric patient, respectively, for instance a bone having an angular deformation to be corrected through the application of the fixator device 10a.

FIG. 3 illustrates a perspective view of the internal fixator device 10b, corresponding to a deformed condition of the internal fixator device 10a, for instance corresponding to an evolution of the implantation of the internal fixator device 10 for an epiphysiodesis intervention, in which the plate is bending-deformed and the fixing screws are spread apart.

As described, the central portion 22 is configured for a bending deformation of the bone plate 20 in the epiphysiodesis intervention. Moreover, the heads 32 of the fixing screws 32 are configured to remain rotationally constrained to the respective holding elements 21, as it will be further described.

As visible, by comparing the deformed configuration of the internal fixator device 10b with the undeformed configuration of the internal fixator device 10a, the stems 31 of the fixing screws 30 are configured to spread apart, thus defining an angle during the bending deformation of the central portion 22 of the plate 20. The fixing screws 30 are locally fixed with respect to the plate 20, so that the expected correction of the alignment of the limb is only due to the deformation of the plate 20 itself, without the heads 32 of the fixing screws 30 substantially rotating (that is, unless there are slight local deformations, which are negligible in practice) with respect to the holding elements 21.

The bending of the bone plate 20 corresponds to a relative rotation of the individual planes of the holding elements 21, which therefore are adapted to allow the fixing screws 30 to spread apart, so as to accommodate the angulation variations that occur during bone growth, allowing better ; tolerability of the implant in epiphysiodesis interventions.

As described, the central portion 22 of the plate 20 is at least partially flexible to allow the bending deformation of the bone plate 20 itself. Meanwhile, the central portion 20 is configured to resist traction, thus ensuring the structural integrity of the bone plate 20 during the traction-bending phase.

The internal fixator device further comprises at least one sensor 40 applied to the central portion 22 of the plate 20. The at least one sensor 40 is configured to measure the bending deformation of the plate 20. The at least one sensor 40 is further configured to wirelessly transmit data representing the bending deformation of the plate 20 to calculate the angle defined by the stems 31 of the fixing screws 30.

In other words, by means of the at least one sensor 40 it is possible to calculate the deformation angle of the plate corresponding to the spreading apart of the stems 31 of the fixing screws, for instance of the deformed configuration 10b with respect to the undeformed configuration 10a of the internal fixator device 10.

In the embodiment of the internal fixator device 10 the at least one sensor 40 comprises a strain gauge sensor 41. A strain gauge sensor is a sensor used to measure the deformation of the objects. A strain gauge is made of a pattern of metal foil on a flexible substrate which may be easily fixed to an object in order to monitor the deformation thereof by measuring a resistance variation.

In an alternative embodiment of the internal fixator device, not represented, the at least one sensor comprises a piezoresistive strain sensor.

The piezoresistive effect is a change in the electrical resistivity of a semiconductor or metal when a mechanical stress is applied. Traditionally, piezoresistive strain sensors are metal-based and have high sensitivities. Most of them are fixed directional sensors and the deformation can only be measured in a specific direction; they have low flexibility/elasticity.

In yet an alternative embodiment of the internal fixator device, not represented, the at least one sensor comprises a piezoelectric strain sensor.

Piezoelectric strain sensors are based on piezoelectric materials, which allow mechanical energy to be converted into electrical energy. Piezoelectric strain sensors have ultrafast response, high sensitivity and low power consumption compared to other types of strain sensors.

Preferably, the at least one sensor 40 is applied to the central portion 22 of the plate 20 by gluing or welding. Alternatively, if allowed by the specific technology, the at least one sensor is at least partially printed directly on a surface of the central portion 22 of the plate 20.

Preferably, the at least one sensor 40 comprises an inductive coupling system configured to wirelessly transmit and non-invasively read the data relating to the bending deformation of the plate 20.

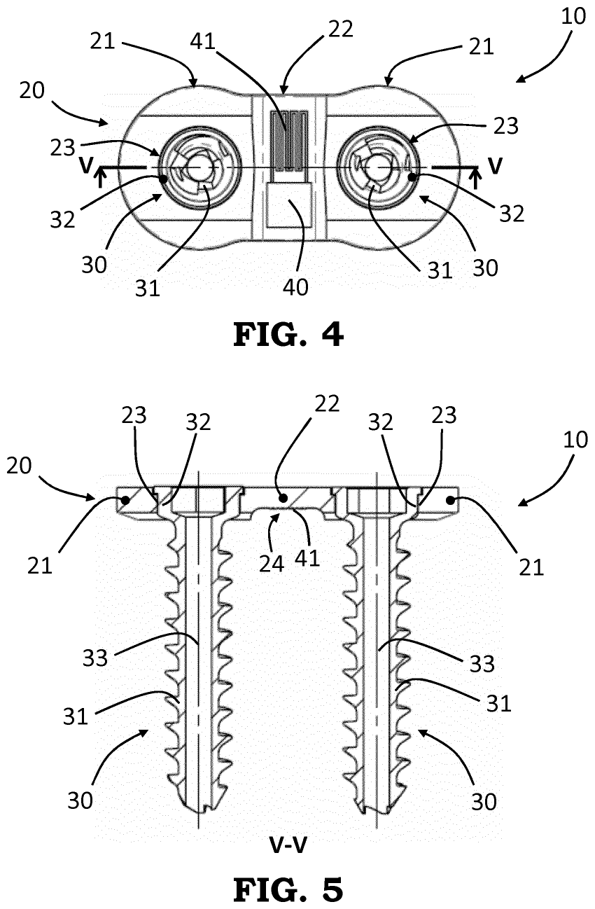

FIG. 4 illustrates a bottom view of the fixator device 10. In this view, it is appreciated that the pair of holding elements 21 and the central portion 22 define a plate 20 that is substantially plane and eight-shaped.

FIG. 5 illustrates a view in section V-V of FIG. 4 of the internal fixator device 10.

In this view it is possible to appreciate that the central portion 22 comprises a raised surface 24 which defines a concavity with respect to the bottom surfaces of the holding elements 21. The bottom surfaces of the holding elements 21 are the ones facing towards the stems 31 of the fixing screws 30.

The raised portion 24, in particular, allows further raising the central portion 22 and generating a bending preferential axis of the plate 20. Moreover, the raised surface 24 is further adapted to limit the pressures on the bone physis when the fixator device 10 is implanted.

In particular, the raised surface 24 is adapted to house the at least one sensor 40, avoiding arranging the sensor on the upper portion of the plate 20 and improving the tolerability of the internal fixator device 10 in the implantation areas.

The internal fixator device according to the present invention is thus more tolerable, in particular when the implant is applied in anatomical regions with poor soft tissue coverage. In this way, it is possible to limit unwanted pressures on the bone physis and thus avoid lateral crushing of the growth cartilage.

In general, it is possible to also appreciate how the plate 20 advantageously has lower perimeter edges which are rounded for greater tolerability.

As visible from the sectional view, each through-hole 23 of the holding elements 21 of the plate 20 comprises a cylindrical internal wall; moreover, each head 32 of the fixing screws 30 comprises a cylindrical external wall configured for a shape mating with the cylindrical internal wall of the through-hole 23.

Preferably, the stem 31 of the fixing screws 30 is self-threading. Moreover, preferably, the stem 31 of the fixing screws 30 is cannulated, i.e. it comprises an axial hole.

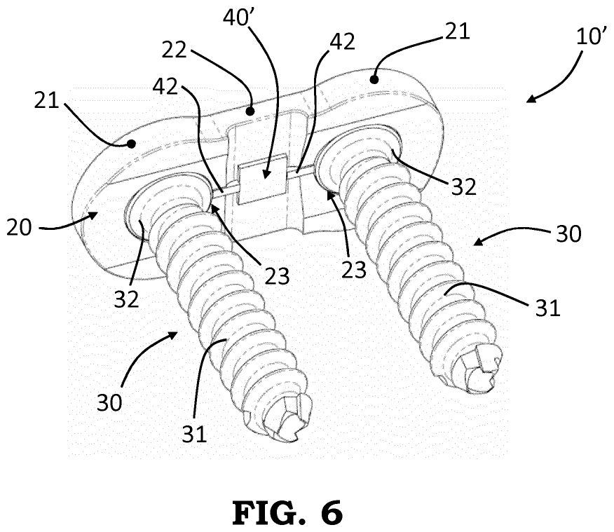

FIG. 6 illustrates a perspective view of a second embodiment of the internal fixator device 10′ for epiphysiodesis intervention.

Compared to the already described embodiment, the internal fixator device 10, the internal fixator device 10′ has at least one sensor 40′ which comprises a capacitive strain sensor. Capacitive strain sensors employ two layers of stretchable conductive films that act as electrodes on either side of an elastomeric dielectric, to form a parallel-plate capacitor.

The at least one sensor 40′ is applied to the central portion by gluing or welding.

In this embodiment, the at least one sensor 40′ comprises a dipole antenna 42 that is configured to wirelessly transmit and non-invasively read the data representing the bending deformation of the plate 20.

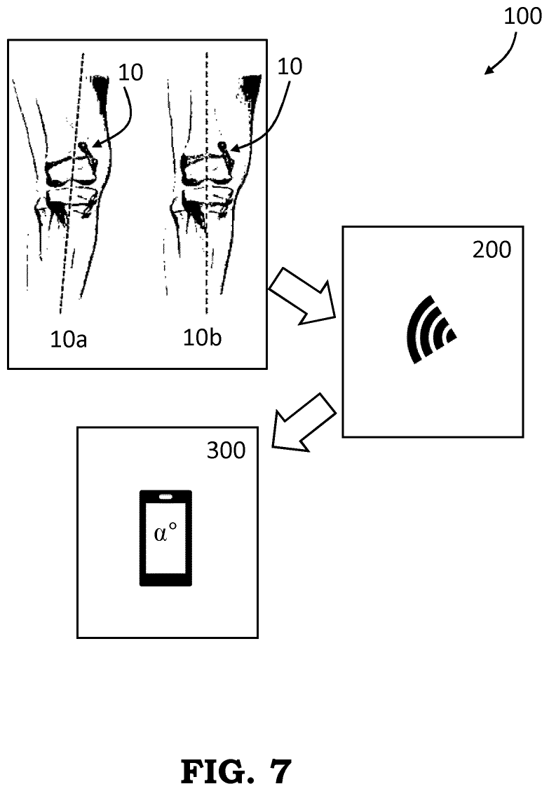

FIG. 7 exemplifies the operation of a monitoring apparatus 100 for epiphysiodesis intervention according to the present invention.

The monitoring apparatus 100 comprises at least one fixator device 10 or 10′, for instance as already described.

The monitoring apparatus 100 further comprises at least one reader 200 configured to wirelessly receive the data representing the bending deformation of the plate 20, transmitted by the at least one sensor 40 or 40′.

The monitoring apparatus 100 further comprises at least one processor 300 configured to process the data representing the bending deformation to calculate the angle defined by the stems 31 of the fixing screws 30 during an evolution of the epiphysiodesis intervention. In particular, the at least one processor 300 comprises a portable electronic device, such as a smartphone.

### Industrial Applicability

The internal fixator device of the present invention represents an advantageous solution to be used as correction means in epiphysiodesis interventions. The present invention further provides the definition of a monitoring apparatus for epiphysiodesis intervention, which exploits the capabilities of the internal fixator device with sensorized plate.

The present invention allows effectively responding to post-operative needs highlighted in the use scenarios, providing measurements of the angular spreading apart of the fixing screws under load and allowing, in principle, measuring uni-planar bending deformations or even on multiple planes if required.

The present invention allows defining a sensorized internal fixator device which is stable for the entire treatment period (up to 4-6 months).

The plate of the internal fixator device of the present invention has an effective integration of the sensor part, so that the detection components do not exceed the thickness of the heads of the fixing screws, to the advantage of tolerability.

The internal fixator device of the present invention allows integrating power supply and data communication solutions directly within/on the plate.

The monitoring apparatus of the present invention provides telemetry solutions and the subcutaneous placement of the transmitter.

The internal fixator device and the monitoring apparatus of the present invention allow defining a solution that can be easily integrated into the epiphysiodesis surgical procedure.

The internal fixator device and the monitoring apparatus of the present invention allow effectively measuring angular spreading apart of the fixing screws from 0° to 30°, with a resolution of at least 1°.

The monitoring apparatus of the present invention allows a measurement of the absolute deformation that can be performed quickly and multiple times (when requested by the surgeon) during the epiphysiodesis intervention period.

Moreover, the internal fixator device of the present invention represents a solution that is substantially insensitive to variation in environmental conditions.

Considering the description herein reported, the person skilled in the art may conceive further changes and variants, in order to meet contingent and specific needs.

It is clear that, where there are no technical incompatibilities evident to the person skilled in the art, the configurations of specific elements described with reference to certain embodiments can be used in other embodiments herein described.

For instance, the specific technologies of the at least one sensor can be adapted to the design requirements, providing multiple sensor components or sensors of different types, also with regard to the wireless data transmission part.

Moreover, for instance, the specific geometries of the plate or of the fixing screws can be adapted to the design requirements, besides what represented in the drawings herein attached.

Therefore, the embodiments herein described are to be intended as illustrative and non-limiting examples of the invention.

1-15. (canceled)

16. An internal fixator device for epiphysiodesis intervention, said internal fixator device comprising:
a bone plate comprising a pair of holding elements and a central portion structurally connecting said pair of holding elements, wherein each of said holding elements comprises a respective through-hole; and
a pair of fixing screws, each of said fixing screws comprising a respective stem configured for fixing to a bone portion and a respective head configured to be held in said respective through-hole;
wherein said central portion is configured for a bending deformation of said bone plate in said epiphysiodesis intervention, said heads of said fixing screws remaining rotationally constrained to the respective said holding elements,
said stems of said fixing screws being configured to spread apart defining an angle in said bending deformation,
wherein said internal fixator device further comprises at least one sensor applied to said central portion and configured to measure said bending deformation, said at least one sensor being further configured to wirelessly transmit data representing said bending deformation in order to calculate said angle defined by said stems.

17. The internal fixator device according to claim 16, wherein said pair of holding elements and said central portion define a substantially planar and eight-shaped plate.

18. The internal fixator device according to claim 16, wherein said central portion comprises a raised surface which defines a concavity with respect to bottom surfaces of said holding elements, said bottom surfaces facing towards said stems, said raised surface being adapted to house said at least one sensor.

19. The internal fixator device according to claim 16, wherein said bone plate is made of a plastic material.

20. The internal fixator device according to claim 19, wherein said plastic material comprises a thermoplastic organic polymer.

21. The internal fixator device according to claim 16, wherein said central portion is at least partially flexible in order to allow said bending deformation of said bone plate.

22. The internal fixator device according to claim 16, wherein said through hole comprises a cylindrical internal wall and wherein said head comprises a cylindrical external wall configured for a shape mating with said cylindrical internal wall.

23. The internal fixator device according to claim 16, wherein said stem of said fixing screws is self-threading.

24. The internal fixator device according to claim 16, wherein said at least one sensor comprises one of the following: a strain gauge sensor, or a capacitive strain sensor, or a piezoresistive strain sensor, or a piezoelectric strain sensor.

25. The internal fixator device according to claim 16, wherein said at least one sensor is applied to said central portion by gluing or welding.

26. The internal fixator device according to claim 16, wherein said at least one sensor is at least partially printed directly on a surface of said central portion.

27. The internal fixator device according to claim 16, wherein said at least one sensor comprises an inductive coupling system configured to wirelessly transmit and non-invasively read said data.

28. The internal fixator device according to claim 16, wherein said at least one sensor comprises a dipole antenna configured to wirelessly transmit and non-invasively read said data.

29. The internal fixator device according to claim 19, wherein said bone plate is made as a single piece.

30. The internal fixator device according to claim 20, wherein said plastic material comprises polyether ether ketone or PEEK.

31. The internal fixator device according to claim 23, wherein said stem of said fixing screws is cannulated.

32. A monitoring apparatus for epiphysiodesis intervention, comprising:
at least one internal fixator device according to claim 15;
at least one reader configured to wirelessly receive said data representing said bending deformation which are transmitted by said at least one sensor; and
at least one processor configured to process said data representing said bending deformation in order to calculate said angle defined by said stems in a development of said epiphysiodesis intervention.

33. The monitoring apparatus according to claim 32, wherein said at least one processor comprises a portable electronic device.

34. The monitoring apparatus according to claim 33, wherein said portable electronic device is a smartphone.
